(19) United States
(12) Patent Application Publication
(10) Pub. No.: US 2005/0101834 A1
(43) Pub. Date: May 12, 2005

(54) Readily implantable sling

(76) Inventor: Bryon L. Merade, Thousand Oaks, CA (US)

Correspondence Address: STETINA BRUNDA GARRED & BRUCKER, 75 ENTERPRISE, SUITE 250, ALISO VIEJO, CA, 92656, US

(21) Appl. No.: 10/947,182

(22) Filed: Sep. 22, 2004

Related U.S. Application Data
(60) Provisional application No. 60/518,930, filed on Nov. 10, 2003.

(51) Int. Cl. A61F 2/00

(52) U.S. Cl. 600/37; 600/30

(57) ABSTRACT

An implantable sling or support having implantation attachment mechanisms pre-attached thereto. Exemplary of such attachment mechanisms include sutures, attachment segments and/or fabric-like materials for facilitating the ingrowth of tissue. The sling/supports are manufactured and packaged with the attachment mechanisms already pre-attached so that the implantable sling can be readily affixed into position during surgery.

### CROSS-REFERENCE TO RELATED APPLICATIONS

The following non-provisional utility patent application claims priority to U.S. Provisional Patent Application Ser. No. 60/518,930 entitled SLING HAVING SUTURES PREATTACHED THERETO, filed on Nov. 10, 2003, the teachings of which are expressly incorporated herein by reference.

### STATEMENT RE: FEDERALLY SPONSORED RESEARCH/DEVELOPMENT

Not Applicable

### BACKGROUND OF THE INVENTION

The present invention is directed to implantable slings, and more particularly, implantable suburethral slings having sutures (e.g., strings, cords, lines, ropes, etc.), implants, tissues, and/or natural or synthetic materials pre-attached thereto or integrally formed thereon to enable the sling to be readily implanted by a surgeon.

The use of slings, and in particular suburethral slings for the treatment of urinary incontinence, are well-known to those skilled in the art. In this regard, such slings provide physical support to the urethra to thus prevent leakage of urine therethrough. The specifics regarding suburethral sling surgical procedures are described in detail in the references of Blaivas, Jerry G., Successful Pubovaginal Sling Surgery, Contemporary Urology, July, 1993; Blaivas, Jerry, G. Treatment of Female Incontinence Secondary To Urethral Damage Or Loss, Urologic Clinics of North America, Vol. 18, No. 2, May, 1991; Raz, Schlomo, Surgical Therapy For Urinary Incontinence, Atlas Of Transvaginal Surgery, W. B. Saunders, 1992, Loughlin, K. R., The Endoscopic Fascial Sling Treatment of Female Urinary Stress Incontinence, J. Urol, 1996, A.P.R.; 155 (4): 1265-7; and Staskin, D. R., et al., The Gore-Tex Sling Procedure For Female Sphincteric Incontinence: Indications, Technique And Results, J. Urol, 1997; 15(5): 295-9, the teachings of which are expressly incorporated herein by reference.

In order to secure such sling into position typically requires that the sling, and more particularly the ends thereof be placed in resident or seated position. To that end, slings may have sutures that are attached by a surgeon to the opposed ends thereof during surgical implantation that serve as the link for attaching the opposed ends of the sling to an anatomical structure, such as the pubic bone, fascia, or some other structure such as Cooper's ligament. Such sutures could serve to allow those skilled in the art to pull the sling into position through suprapubic, vaginal, obturator, or other approaches. Anchoring devices are further frequently deployed that are operative to secure the ends of the sling, typically by sutures extending from the opposed ends of the sling at a target site within the patient's body. Exemplary of the techniques utilized to secure the sutures extending from the opposed ends of the sling into position are disclosed in U.S. Pat. No. 6,200,330 to Benderev, et al. entitled Systems for Securing Sutures, Grafts and Soft Tissue to Bone and Periosteum, issued Mar. 13, 2001, the teachings of which are expressly incorporated herein by reference. Alternatively, slings may be formed such that the free ends of the sling are merely allowed to rest in position with the intermediate portion being operative to provide urethral support. Exemplary of such products include Gynecare TVT, produced by Johnson & Johnson of New Brunswick, N.J.

In all such surgical procedures, however, it is necessary for the opposed ends of the sling to be manipulated such that the sling remains optimally positioned within the patient. As discussed above, however, to secure the sling into position requires that the respective ends of the sling be provided with some sort of mechanism that ensures that the same can be properly implanted, which is likewise well-known to add substantial time, effort and expense to the surgical procedure. For example, to attach a suture line to such opposed ends of the sling is time consuming and requires precise surgical skill. Moreover, to the extent the suture is not adequately secured to the opposed end of the sling, a possibility exists that the suture may become detached from the sling, which would thus defeat the purpose of the surgical procedure. Moreover, attaching the sutures to the sling, if done improperly, could damage the sling sought to be implanted thus requiring a new sling to be utilized. These same complications also arise to the extent the opposed ends of the sling are sought to be directly affixed to an implant or some other type of material to facilitate the ingrowth of tissue. Such events can substantially add to the time, expense, and potential patient discomfort associated with the surgical procedure, especially if the sling to be utilized during such surgical procedure is derived from tissue harvested from the patient, which may necessarily require that additional tissue be harvested.

Accordingly, there is a substantial need in the art for a readily implantable sling, and in particular an implantable suburethral sling that is already provided with ends having means formed or attached thereon that provide for quick and easy surgical implantation.

### BRIEF SUMMARY OF THE INVENTION

The present invention specifically addresses and alleviates the above-identified deficiencies in the art. Specifically, the present invention is directed to an implantable slings and support structures having implantation attachment structures pre-attached thereto or pre-formed thereon. In this regard, such implantation attachment structures may comprise any of a variety of structures, including sutures, grafts, tissues, and any other type of natural and synthetic material that facilitates the ability of the sling to be implanted and secured into position. The sling may comprise any implantable sling known in the art, and expressly includes suburethral slings operatively positionable beneath the urethra of a patient. Likewise, such sling may include implants for cystocele, rectocele, enterocele, prolapse and/or any combination thereof.

Per conventional slings, the sling of the present invention may be fabricated from natural or synthetic tissue, and, in the case of suburethral slings, will preferably be formed as an elongate strip having opposed ends. The attachment structures, such as conventional sutures, strings, cords, lines, or any other structures known in the art, are pre-attached to the sling, and preferably at the respective opposed ends of the sling, with the resultant implant thereafter being packaged as a unit. Alternatively, the attachment structures are integrally formed as part of the sling. During a surgical procedure, and in particular pubovaginal sling surgery, the sling having the pre-attached attachment structures will be removed from the packaging and secured into position within a patient per conventional surgical procedures. In alternative embodiments directed to cystocele, rectocele, enterocele, prolapse and the like, the implantable sling will be pre-formed to have the desired configuration for use in supporting the appropriate anatomical structure but will also have one or more attachment structures pre-attached thereto or pre-formed thereon to facilitate implantation. Advantageously, by having the attachment means pre-attached in advance and in a manner that assures that such attachment structures are properly affixed to the sling/support structure, considerable time and money is conserved during the surgical procedure.

With respect to the fabrication of the sling/support structure with pre-attached attachment means of the present invention, the same can readily be made and packaged per conventional manufacturing techniques. Along these lines, it is contemplated that the slings with the attachment structures attached thereto will be manufactured and/or fabricated in a sterile environment and packaged such that the sling implants remain in a sterile condition until such time as the same are utilized for a surgical procedure.

### BRIEF DESCRIPTION OF THE DRAWINGS

These as well as other features of the present invention will become more apparent upon reference to the drawings.

FIG. 1 is a top perspective view of an elongate, implantable sling having opposed ends and showing dedicated sutures pre-attached to the respective ones of the opposed ends.

FIG. 2 is a top perspective view of an elongate, implantable sling having opposed ends and showing elongate attachment strips of anchoring material pre-attached to respective ones of the opposed ends of said sling.

FIG. 3 is a top perspective view of implantable sling of FIG. 2 wherein said attachment strips are formed integral with the implantable sling.

FIG. 4 is a top perspective view of an elongate, implantable sling constructed in accordance with another preferred embodiment having materials attached to the opposed ends thereof for facilitating tissue ingrowth.

FIG. 5 is a top perspective view of an implantable sling/support structure operative to concurrently treat incontinence and effectuate cystocele repair.

FIG. 6 is a top perspective view of an implantable sling/implant for effectuating incontinence and effectuating either enterocele and/or cystocele repair.

FIG. 7 is a top perspective view of an implantable sling/support structure operative to concurrently effectuate cystocele and enterocele and/or rectocele repair.

### DETAILED DESCRIPTION OF THE INVENTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

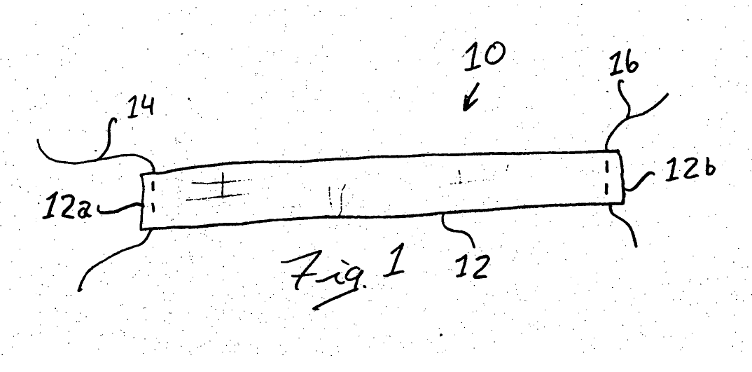

Referring now to the Figures, and initially to FIG. 1, there is shown an implant 10, and more particularly an implantable sling having pre-attached sutures thereto as constructed in accordance with a preferred embodiment of the present invention. As illustrated, such implant 10 comprises a sling portion 12 that, as per conventional slings and in particular suburethral slings, is fashioned to have a generally elongate configuration defining a first end 12a and a second end 12b. As will be readily appreciated by those skilled in the art, such sling portion 12 may be fabricated from natural tissues, such as tissues harvested from a donor or cadaver, or even the patient within which the sling 12 will be implanted. Alternatively, the sling portion 12 may be fabricated from any of a variety of well-known synthetic materials useful for fabricating implants and the like or from a combination of both natural and synthetic materials. Additionally, the sling portion 12 may be configured per any of a variety of slings currently utilized in implantable sling applications. Along these lines, it is expressly contemplated that the sling 12 will be particularly well suited for use in pubovaginal sling surgery; however, it is contemplated that the implant 10 of the present invention may be utilized in any other surgical application where a sling must necessarily be deployed. Along these lines, and as discussed more fully below, it is contemplated that the sling 12 may be fashioned as a combination sling or otherwise to serve as a support structure to treat pelvic organ prolapse or otherwise effectuate either cystocele, rectocele and/or enterocele repair. Accordingly, it should be understood that the term sling and/or support structure should be construed as broadly as possible.

On the respective ends 12a, 12b of the sling portion 12 of the implant 10 are pre-attached sutures 14, 16. More particularly, suture 14, which may take the alternative form of either a rope, cord, line, string or any other like structure, is affixed to end 12a of sling portion 12 of implant 10 whereas suture 16 is attached to end 12b of sling portion 12 or the implant 10. Such sutures 14, 16 may take any of a variety of suture materials known in the art. In addition, such sutures 14, 16 may be attached to the respective opposed ends 12a, 12b of the sling portion 12 of implant 10 via any of a variety of known techniques, including the stitch attachment as illustrated. It is likewise contemplated that the length of sutures 14, 16 and be integral to the design rather than a separate attachment may be selectively chosen to thus enable the implant 10 to be more easily implanted into position, and may have a length that is selectively chosen depending upon the specific site to which the sutures 14, 16 are attached, as well as whether or not any additional slack may be necessary in such sutures 14, 16 as may be desired for a given medical procedure.

It is further contemplated that the implant 10 may be fabricated via any of a variety of well-known techniques known in the art. In this regard, sling portion 12 may take the form of any conventional sling design known in the art. Likewise, the attachment of the sutures to such sling may be effectuated per conventional sterile manufacturing practices, also well-known in the art. Along these lines, it is contemplated that the implant 10 may be manufactured in a sterile environment and thereafter packaged to maintain such sterile condition until such time as the same are utilized for implantation during a surgical procedure. Accordingly, the implants 10 of the present invention not only provide a readily useable implant that may be more efficiently utilized during a surgical procedure, but also can possess a long shelf life that can help ensure an adequate supply of such implants as may be needed to supply a particular demand, as may be encountered by a hospital or other type of out-patient surgical facility.

Referring now to FIGS. 2-4, there are shown three additional embodiments of the readily implantable slings of the present invention. With respect to the embodiment depicted in FIG. 2, there is shown an elongate sling 12 that, as per the embodiment depicted in FIG. 1, is formed as an elongate segment having opposed ends 12a, 12b. Formed upon such opposed ends 12a, 12b are attachment strips or segments 18, 20. More particularly, attachment strip/segment 18 is affixed to end 12a, whereas second attachment strip/segment 20 is attached to second end 12b of strip 12. The attachment strips may take any of a variety of configurations known in the art that are operative to facilitate the attachment of the sling 12 into position. As illustrated, such attachment strips/segments 18, 20 are formed as elongate members having flat or string-like portions 18a, 20b extending distally from the opposed ends 12a, 12b to which the strips/segments 18, 20 are attached. In this regard, such portions 18a, 20b will be operative to be surgically manipulated so that the same can be anchored into position or otherwise secured in place to thus enable the sling 12 to be optimally positioned and impart the desired degree of support. Such attachment strips/segments 18, 20 may be fabricated from any of a variety of materials well-known to those skilled in the art, which may include either natural or synthetic material, tissues, grafts and the like as would be readily be known by those skilled in the art. In accordance of the principles of the present invention, the attachment strips/segments 18, 20 will be pre-attached to the opposed ends 12, 12b of the sling 12. More particularly, attachment strip/segment 18 will be attached to end 12a at juncture 22, which can be accomplished by any of a variety of techniques well-known in the art, including stitching the anchoring strip/segment 18 to the end 12a of sling 12. A similar attachment will be made for the anchoring strip/segment 20 made at juncture 24 with end 12b of sling 12.

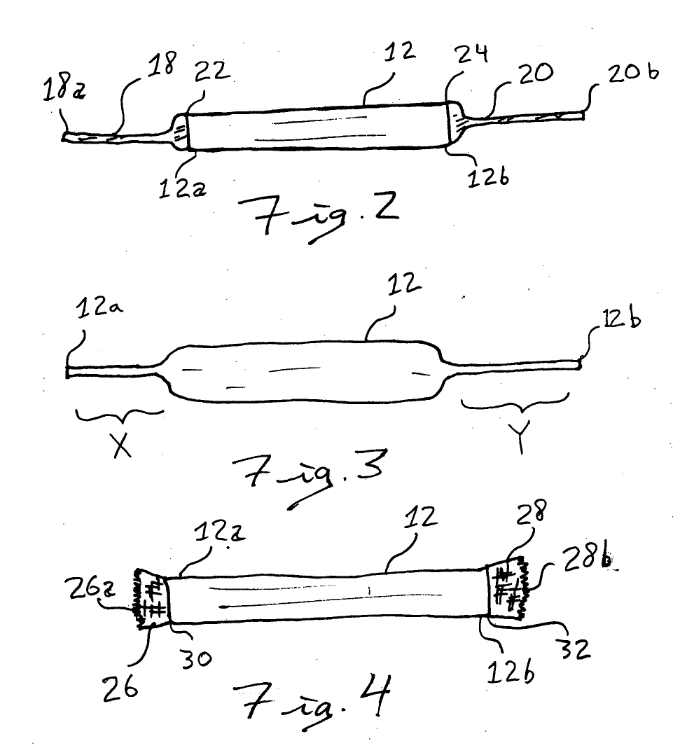

Referring now to FIG. 3, there is shown an alternative embodiment wherein the attachment strips/segments depicted in FIG. 2 are integrally formed as part of sling 12. As shown, the sling 12 will be fabricated from a unitary piece of material such that the same defines a sling body 12, which will be operative to be implanted and provide a necessary degree of support to an anatomical structure, with the opposed ends 12a, 12b extending therefrom and defining the elongate strips/segments that are otherwise separately pre-attached in the embodiment depicted in FIG. 2. As will be appreciated by those skilled in the art, such attachment strip/segment portions, whether fabricated from a unitary piece of material as depicted in FIG. 3, or separately pre-attached in the embodiment depicted in FIG. 2, may be provided to have any of a variety of shapes that may be operative to facilitate the ability of the surgeon to readily affix the sling 12 into position. As will be readily appreciated, and as shown in FIG. 3, the length of such attachment strip/segment portions may be formed to have a desired length, represented by the letters X and Y, respectively. As such, it will be readily recognized by those skilled in the art that the slings 12 of the present invention may be pre-manufactured to attain such optimal specifications. Moreover, with respect to the embodiment depicted in FIG. 3, it will be readily appreciated by those skilled in the art that such sling 12, as well as attachment strips/segments extending therefrom to define the ends 12a, 12b of such embodiment may be fabricated from any material well-known to those skilled in the art, whether it me natural or synthetic.

Referring now to FIG. 4, there is shown yet a further embodiment of the present invention wherein the sling 12 is provided with frayed cloth portions 26, 28 attached to the respective ends 12a, 12b thereof. According to such embodiment, such portions 26, 28 will formed from a cloth-like material that facilitates the ingrowth of tissue, to thus enable the sling to become permanently affixed into position over time. Per the embodiment illustrated in FIG. 2, such cloth portions 26, 28 will be affixed to the respective ends 12a, 12b via any of a variety of techniques known in the art, such as via sutures and the like, at junctures 30, 32, respectively. To facilitate the ingrowth of tissue, such portions 26, 28 will be provided with frayed ends 28a, 28b, respectively, that, as is well-known in the art, provide a greater surface area about which scarring and tissue formation can develop.

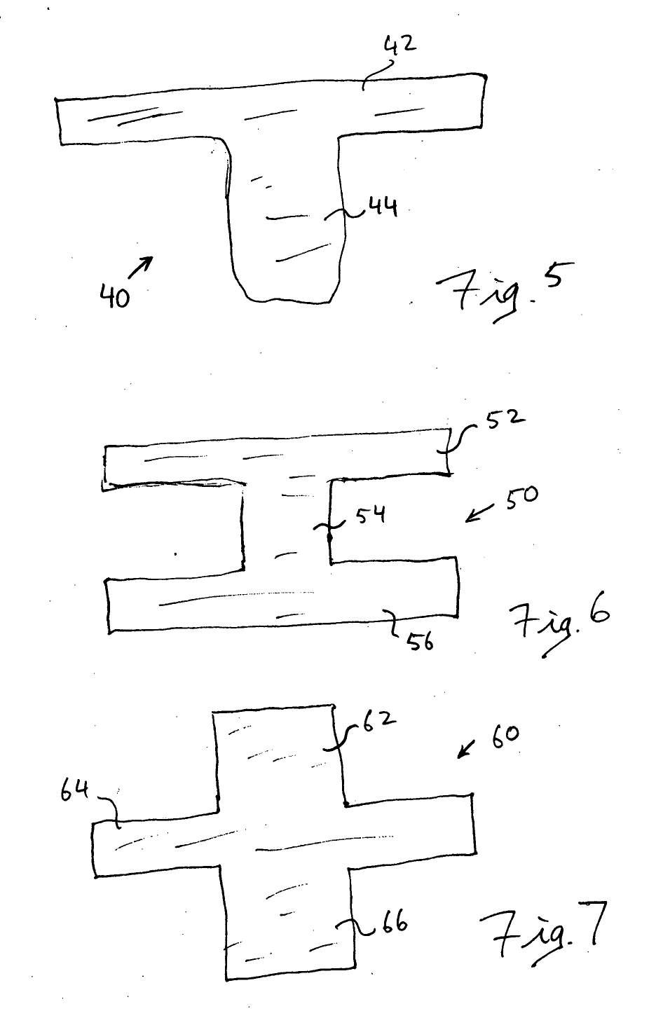

Referring now to FIGS. 5-7, and initially to FIG. 5, there is shown three further embodiments of implantable sling/support structures deemed to fall within the scope of the invention that are operative to treat two or more conditions, such as prolapse or incontinence, or otherwise effectuate cystocele, enterocele and/or rectocele repair. Referring initially to FIG. 5, there is shown an implant 40 operative to treat incontinence, via sling portion 42, as well as serve to effectuate cystocele repair via support portion 44. Along these lines, such implant 40 may take the form of those implants disclosed and claimed in Applicant's pending U.S. patent application Ser. No. 10/684,861, filed Oct. 14, 2003, entitled IMPLANTABLE SLING HAVING BLADDER SUPPORT, the teachings of which are expressly incorporated herein by reference. Such implant 40 will be provided with one or more attachment structures, whether it be sutures, materials for facilitating ingrowth and/or elongate portions either attached thereto or integrally formed thereon that can be manipulated by a surgeon during implantation.

Referring to FIG. 6, there is shown an implant 50 that is operative to treat incontinence via elongate strip portion 52, as well as provide additional support via additional portions 54 and elongate strip portion 56. With respect to the latter two portions, section 54 may be operative to effectuate either cystocele or enterocele repair and elongate portion 56 may be operative to effectuate enterocele repair. Likewise, per all the embodiments herein, sutures, materials for facilitating ingrowth, or elongate portions can be formed anywhere about the implant 50 as may be desired to facilitate the surgical placement and fixation of such implant 50.

In yet another embodiment depicted in FIG. 7, there is shown an implant 60 having a first enlarged portion 62, an intermediate strip portion 64, and a second enlarged portion 66 that is configured to concurrently effectuate cystocele and/or enterocele on one hand, and rectocele on the other hand. In this respect, support portions 62, 66 define areas that can support the bladder, intestine, or rectum. Elongate strip portion 64 is operative to facilitate placement of such implant 60. Again, such implant 60 will be provided with sutures or any other attachment structure discussed herein to facilitate surgical placement of such implant 60.

Additional modifications and improvements of the present invention may also be apparent to those of ordinary skill in the art. Along these lines, it is expressly contemplated that any type of sling having pre-formed or pre-attached members, sutures, and/or segments formed upon the opposed ends of such sling for facilitating surgical attachment, placement, or positioning is expressly considered to fall within the scope and spirit of the present invention. Thus, the particular combination of parts and steps described and illustrated herein is intended to represent only certain embodiments of the present invention, and is not intended to serve as limitations of alternative devices and methods within the spirit and scope of the invention.

1. An implant comprising:
a) an elongate sling having first and second opposed ends;
b) a first suture pre-attached to said first opposed end of said sling;
c) a second suture pre-attached to said second opposed end of said sling; and
d) wherein said sling having said first and second pre-attached sutures thereto are held within a sterile packaging.

2. The implant of claim 1 wherein said sling portion of said implant is operatively positionable beneath a urethra of a patient.

3. An implant comprising:
a) an elongate sling having first and second ends;
b) a first attachment mechanism pre-attached to said first opposed end of said sling;
c) a second attachment mechanism pre-attached to said second opposed end of said sling; and
d) wherein said sling having said first and second pre-attached attachment mechanisms are held within a sterile packaging.

4. The implant of claim 3 wherein said first and second attachment mechanisms are selected from the group consisting of sutures, attachment segments and material for facilitating the ingrowth of tissue.

5. The implant of claim 4 wherein said attachment segment is fabricated from a synthetic material.

6. The implant of claim 4 wherein said attachment segment is fabricated from a natural material.

7. The implant of claim 6 wherein said natural material comprises a tissue.

8. The implant of claim 6 wherein said natural material comprises a graft.

9. The implant of claim 3 wherein said first attachment mechanism comprises an attachment segment integrally formed with said first opposed end of said sling.

10. The implant of claim 9 wherein said second attachment mechanism comprises an attachment segment integrally formed upon said second opposed end of said sling.

11. The implant of claim 3 wherein said elongate sling is fabricated form a natural material, synthetic material or combinations thereof.

12. An implant comprising:
a) a support structure operative to support at least two anatomical structures selected from the group consisting of the urethra, the bladder, a portion of the small intestine, and rectum of a patient;
b) at least one attachment mechanism pre-attached to said support; and
c) wherein said support having said at least one pre-attached attachment mechanism are held within a sterile packaging.

13. The implant of claim 1 wherein said sling of said implant is operative to effectuate cyctocele repair.

14. The implant of claim 1 wherein said sling of said implant is operative to effectuate enterocele repair.

15. The implant of claim 1 wherein said sling of said implant is operative to effectuate rectocele repair.

16. The implant of claim 1 wherein said sling of said implant is operative to effectuate treatment of prolapse.

17. The implant of claim 1 wherein said sling of said implant is operative to effectuate treatment of urinary incontinence.
